(19) United States
(12) Patent Application Publication
(10) Pub. No.: US 2024/0310322 A1
(43) Pub. Date: Sep. 19, 2024

(54) DIFFUSION-BASED ELECTROCHEMICAL SENSORS

(71) Applicant: Robert Bosch GmbH, Stuttgart (DE)

(72) Inventors: Young Shik SHIN, Mountain View, CA (US); Nadezda FOMINA, Redwood City, CA (US); Christopher JOHNSON, San Carlos, CA (US); Armin DARVISH, Daly City, CA (US); Gabrielle VUKASIN, San Jose, CA (US); Gary YAMA, Mountain View, CA (US); Christoph LANG, Sunnyvale, CA (US)

(21) Appl. No.: 18/185,124

(22) Filed: Mar. 16, 2023

(51) Int. Cl. G01N 27/416

(52) U.S. Cl. CPC G01N 27/416

(57) ABSTRACT

A target molecule detection method includes contacting oxidation and reduction electrodes with a solution having redox tags. The method further includes applying a first oxidation voltage pulse to the oxidation electrode when the solution does not contain the target molecules to obtain a first oxidation current profile, applying a second oxidation voltage pulse to the oxidation electrode when the solution contains the target molecules to obtain a second oxidation current profile, applying a first reduction voltage pulse to the reduction electrode when the solution does not contain the target molecules to obtain a first reduction current profile, and applying a second reduction voltage pulse to the reduction electrode when the solution contains the target molecules to obtain a second reduction current profile. The method also includes detecting the target molecules in response to one or more electrical properties of the first and second oxidation and reduction current profiles.

### TECHNICAL FIELD

The present disclosure relates to diffusion-based electrochemical sensors. The present invention includes embodiments relating to target molecule diffusion-based electrochemical sensor systems and sensing methods using diffusion mechanisms of bound target molecules between an oxidation electrode and a reduction electrode.

### BACKGROUND

Diffusion is a physical process that reflects movement of molecules in a fluid in a random fashion following a concentration gradient. The rate of diffusion of a molecule may depend on the mass of the molecules, the amount of thermal energy of the molecules, and/or environmental factors such as temperature and/or viscosity. As a general matter, heavier molecules move slower than lighter molecules by diffusion. Two molecules binding together form a binding complex. As a relative matter, the binding complex moves slower than when the two molecules were separate. Diffusion mechanisms have been used in the electrochemical detection of redox molecules in solutions.

### SUMMARY

According to one embodiment, a target molecule diffusion-based electrochemical sensor system is disclosed. The target molecule diffusion-based electrochemical sensor system includes a chamber configured to contain a solution having redox tags and capture agents bound to the redox tags. The redox tags are present in the solution in a reduced state and/or an oxidized state. The capture agents are configured to bind to target molecules introduced into the solution. The target molecule detection system further includes an oxidation electrode configured to oxidize the redox tags present in the solution in the reduced state into the oxidized state. The oxidation electrode is configured to output a first oxidation current profile in response to a first oxidation voltage pulse applied to the oxidation electrode when the solution does not contain the target molecules. The oxidation electrode is configured to output a second oxidation current profile in response to a second oxidation voltage pulse applied to the oxidation electrode when the solution contains the target molecules. The target molecule diffusion-based electrochemical sensor system further includes a reduction electrode spaced apart from the oxidation electrode and configured to reduce the redox tags present in the solution in the oxidized state into the reduced state. The reduction electrode is configured to output a first reduction current profile in response to a first reduction voltage pulse applied to reduction electrode when the solution does not contain the target molecules. The reduction electrode is configured to output a second reduction current profile in response to a second reduction voltage pulse applied to the reduction electrode when the solution contains the target molecules. One or more electrical properties of the first and second oxidation current profiles and the first and second reduction current profiles are indicative of a presence of the target molecules. The initial state of the redox tag may depend on the specific characteristics of the redox tag. If the redox tag is stable in the reduced state, the initial state is the reduced state only, and vice versa. If the initial stable state of the redox tag is in the reduced state, the signaling process starts from the oxidation electrode. If the initial stable state of the redox tag is in the oxidized state, then the signaling process starts from the reduction electrode.

In another embodiment, a target molecule diffusion-based electrochemical sensing method is disclosed. The method includes contacting an oxidation electrode and a reduction electrode with a solution having redox tags and capture agents bound to the redox tags. The redox tags are present in the solution in a reduced state and/or an oxidized state. The capture agents are configured to bind to target molecules introduced into the solution. The method further includes applying a first oxidation voltage pulse to the oxidation electrode when the solution does not contain the target molecules to obtain a first oxidation current profile, applying a second oxidation voltage pulse to the oxidation electrode when the solution contains the target molecules to obtain a second oxidation current profile, applying a first reduction voltage pulse to the reduction electrode when the solution does not contain the target molecules to obtain a first reduction current profile, and applying a second reduction voltage pulse to the reduction electrode when the solution contains the target molecules to obtain a second reduction current profile. The method also includes detecting the target molecules in response to one or more electrical properties of the first and second oxidation current profiles and the first and second reduction current profiles. The initial state of the redox tag may depend on the specific characteristics of the redox tag. If the redox tag is stable in the reduced state, the initial state is the reduced state only, and vice versa. If the initial stable state of the redox tag is in the reduced state, the signaling process starts from the oxidation electrode. If the initial stable state of the redox tag is in the oxidized state, then the signaling process starts from the reduction electrode.

In yet another embodiment, a target molecule diffusion-based electrochemical sensing method is disclosed. The method includes contacting an oxidation electrode and a reduction electrode with a solution having redox tags and capture agents bound to the redox tags. The redox tags are present in the solution in a reduced state and/or an oxidized state. The capture agents are configured to bind to target molecules introduced into the solution. The method further includes applying a first oxidation voltage pulse to the oxidation electrode when the solution does not contain the target molecules to obtain a first oxidation current profile, applying a second oxidation voltage pulse to the oxidation electrode when the solution contains the target molecules to obtain a second oxidation current profile, applying a first reduction voltage pulse to the reduction electrode when the solution does not contain the target molecules to obtain a first reduction current profile, and applying a second reduction voltage pulse to the reduction electrode when the solution contains the target molecules to obtain a second reduction current profile. The method also includes detecting the target molecules in response to a first difference between the first and second oxidation current profiles and/or a second difference between the first and second reduction current profiles. The initial state of the redox tag may depend on the specific characteristics of the redox tag. If the redox tag is stable in the reduced state, the initial state is the reduced state only, and vice versa. If the initial stable state of the redox tag is in the reduced state, the signaling process starts from the oxidation electrode. If the initial stable state of the redox tag is in the oxidized state, then the signaling process starts from the reduction electrode.

### BRIEF DESCRIPTION OF THE DRAWINGS

FIG. 1 depicts a schematic diagram of an electrochemical detection system with an oxidation electrode, a reduction electrode, and a passivation layer.

FIG. 2A depicts a schematic diagram of reaction and diffusion mechanisms of redox tags in the electrochemical detection system of FIG. 1.

FIG. 2B depicts a graph of an oxidation voltage pulse applied to an oxidation electrode (M1) and an oxidation potential of the oxidation electrode.

FIG. 2C depicts a graph of a reduction voltage pulse applied to a reduction electrode (M2) and a reduction potential of the reduction electrode.

FIG. 2D depicts a graph of an oxidation current profile at the oxidation electrode in response to the oxidation voltage pulse shown in FIG. 2B.

FIG. 2E depicts a graph of a reduction current profile at the reduction electrode in response to the reduction voltage pulse shown in FIG. 2C.

FIG. 3A depicts a schematic view of reaction and diffusion mechanisms of redox tags in an electrochemical detection system where target molecules are added to a solution.

FIG. 3B depicts a graph of an oxidation voltage pulse applied to an oxidation electrode (M1) and an oxidation potential of the oxidation electrode.

FIG. 3C depicts a graph of a reduction voltage pulse applied to a reduction electrode and a reduction potential of a reduction electrode.

FIG. 3D depicts a graph of an oxidation current profile at the oxidation electrode in response to the oxidation voltage pulse in FIG. 3B.

FIG. 3E depicts a graph of a reduction current profile at the reduction electrode in response to the reduction voltage pulse in FIG. 3C.

FIG. 4A depicts a schematic view of an electrochemical detection system including a microchannel partially enclosed by first and second valves connected thereto.

FIG. 4B depicts a schematic, perspective view of an electrochemical detection system including a base and an array of microwells extending into the base.

FIG. 4C depicts a magnified, isolated, perspective view of a microwell in the array of microwells.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “substantially,” “generally,” or “about” means that the amount or value in question may be the specific value designated or some other value in its neighborhood. These terms may be used to modify any numeric value disclosed or claimed herein. Generally, the term “about” denoting a certain value is intended to denote a range within ±5% of the value. As one example, the phrase “about 100” denotes a range of 100±5, i.e., the range from 95 to 105. Generally, when the term “about” is used, it can be expected that similar results or effects according to the invention can be obtained within a range of +5% of the indicated value. The term “substantially” may modify a value or relative characteristic disclosed or claimed in the present disclosure. In such instances, “substantially” may signify that the value or relative characteristic it modifies is within +0%. 0.1%, 0.5%, 1%, 2%, 3%, 4%, 5% or 10% of the value or relative characteristic.

It should also be appreciated that integer ranges explicitly include all intervening integers. For example, the integer range 1 to 10 explicitly includes 1, 2, 3, 4, 5, 6, 7, 8, 9, and 10. Similarly, the range 1 to 100 includes 1, 2, 3, 4, . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

As used herein, the term “and/or” means that either all or only one of the elements of said group may be present. For example, “A and/or B” means “only A, or only B, or both A and B”. In the case of “only A”, the term also covers the possibility that B is absent, i.e. “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The description of a group or class of materials as suitable for a given purpose in connection with one or more embodiments implies that mixtures of any two or more of the members of the group or class are suitable. Description of constituents in chemical terms refers to the constituents at the time of addition to any combination specified in the description and does not necessarily preclude chemical interactions among constituents of the mixture once mixed. First definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

Electrochemical detection of redox molecules may be used for detection when capture agents are labeled with redox tag molecules within a solution. According to current proposals, these electrochemical detection processes require washing of the solution or changing the solution. When the capture agents with the redox tags are present in a solution sample, the capture agents with the redox tags contribute to electrical signals from oxidation and reduction electrodes through redox reactions. Oxidation makes the molecule active for signaling purposes when the redox tag is stable in its reduction form. If the redox tag is stable in its oxidation form, reduction is the first step to obtain a signal. Some of the oxidized molecules may reach the other electrode and become reduced. Thus, oxidation and reduction current may be measured from the first and second electrodes.

When target molecules are added, there will be a dilution effect if the target molecules are in a pure buffer. To maintain the same concentration of the redox tag molecules, the target molecule sample may be prepared with the same amount of the redox tag molecules (e.g., a 1:1 mixture of the target molecules and 2× redox tag-labeled capture agents). The dilution factor may be considered for quantification. When target molecules are added, the target molecules form a heavier complex with a capture agent, thereby changing a diffusion rate. In this case, (1) the free capture agent and (2) the bound complex of the target molecules and the capture agents provide two sources for redox signaling. A current proposal uses an interdigital array electrode to measure a diffusion coefficient of a molecule.

However, the current proposals do not measure the redox current profiles from both of an oxidation electrode and a reduction electrode when voltage pulses (e.g., different voltage pulses) are applied to the oxidation electrode and the reduction electrode. A target molecule detection system and method are desired where redox current profiles from voltage pulses (e.g., different voltage pulses) applied to an oxidation electrode and a reduction electrode are measured and the redox current profiles are representative of the presence and/or amount of the target molecules in a solution.

One or more embodiments disclose a method to detect the presence of target molecules when they bind to capture agents. One or more binding complexes are formed when the target molecules bind to the capture agents. The formation of one or more of the binding complexes changes a diffusion behavior characteristic. The change in diffusion behavior characteristic may be detected, thereby detecting the presence of the target molecules (e.g., DNA, protein, and small molecules).

One or more embodiments disclose a system to detect the presence of target molecules. The target molecule detection system includes first and second electrodes (e.g., an oxidation electrode and a reduction electrode). The first and second electrodes may act as a set. In one or more embodiments, the target molecule detection system may include multiple sets of electrode pairs where each electrode pair may have different separation distances and/or different electrode designs.

The target molecule detection system may include capture agents. The capture agents include redox tags configured to provide electrochemical signals from the first and second electrodes. An initial redox signal may serve as a baseline when a sample solution contains capture agents only. Upon the addition of target molecules to the sample solution, a binding complex is formed with at least one of the capture agents and at least one of the target molecules. The formation of the binding complex introduces a change in a diffusion behavior characteristic (e.g., a diffusion rate through the sample solution including the target molecules). A change in the diffusion rate alters a redox current profile, which is reflective of the amount of a target molecule present in the sample molecule. In one or more embodiments, the detection system may operate without a washing step or a solution change because detection is based on the change of the diffusion behavior characteristic of the capture agents.

One or more embodiments are directed to target molecule detection systems and methods for sensing target molecules in a solution phase by measuring electrical parameters for the electrochemical redox signaling to determine a diffusion rate change. The diffusion rate change may be used to detect a presence of the target molecules.

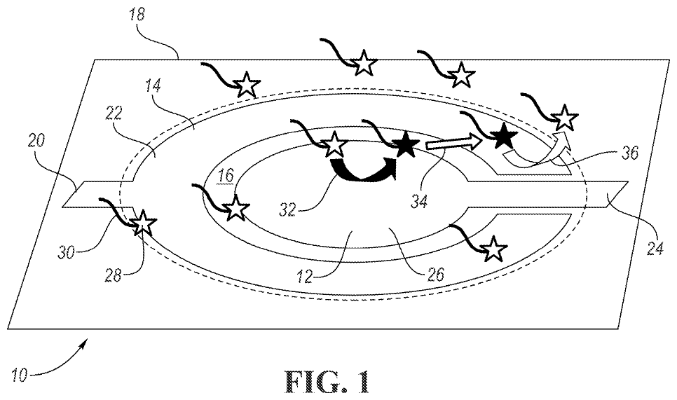

FIG. 1 depicts a schematic diagram of electrochemical detection system 10 with oxidation electrode 12, reduction electrode 14 and passivation layer 18. In one or more embodiments, passivation layer 18 is configured to protect oxidation electrode 12 and reduction electrode 14 from corrosion or oxidation and define the active portion of the electrode, which may enhance the accuracy. Oxidation electrode 12 may be referred to as M1, and reduction electrode 14 may be referred to as M2. Oxidation electrode 12 and reduction electrode 14 may be formed of platinum, gold, any electrically conductive indium tin oxide (ITO) material, and a metal oxide. Oxidation and reduction electrodes 12 and 14 are spaced apart from each other by spaced apart region 16 such that oxidation and reduction electrodes 12 and 14 are not touching each other. Oxidation electrode 12 includes lead portion 24 connected to an electrical source and sensing portion 26 having a curved thickness. Reduction electrode 14 includes lead portion 20 connected to the electrical source and sensing portion 22 having a solid curved region. As shown in FIG. 1, sensing portion 22 of reduction electrode 14 partially surrounds sensing portion 26 of oxidation electrode 12 and is spaced apart therefrom by spaced apart region 16.

Oxidation and reduction electrodes 12 and 14 may be formed of a flexible material. The type of the flexible material may be chosen such that spaced apart region 16 remains fixed and the area of each of oxidation and reduction electrodes 12 and 14 are maintained constant. The area of each of the oxidation and reduction electrodes may be in a nanometer scale to a centimeter, and in some embodiments, in a micrometer range. Oxidation and reduction electrodes 12 and 14 may be separated by a fixed distance. The fixed distance of each of the oxidation and reduction electrodes may be in a nanometer scale to a centimeter, and in some embodiments, in a micrometer range. In one or more embodiments, the type of the flexible material is selected such that the material remains stable under multiple rounds of electrochemical measurements.

As shown in FIG. 1, electrochemical detection system 10 includes a volume of solution. The solution may be comprised of any biological buffer solution. Electrochemical detection system 10 may form a diffusion-based, no-wash sensing mechanism. The solution includes redox tags 28 represented by stars and capture agents 30 represented by curved portions. As shown in FIG. 1, redox tags 28 are bound to capture agents 30.

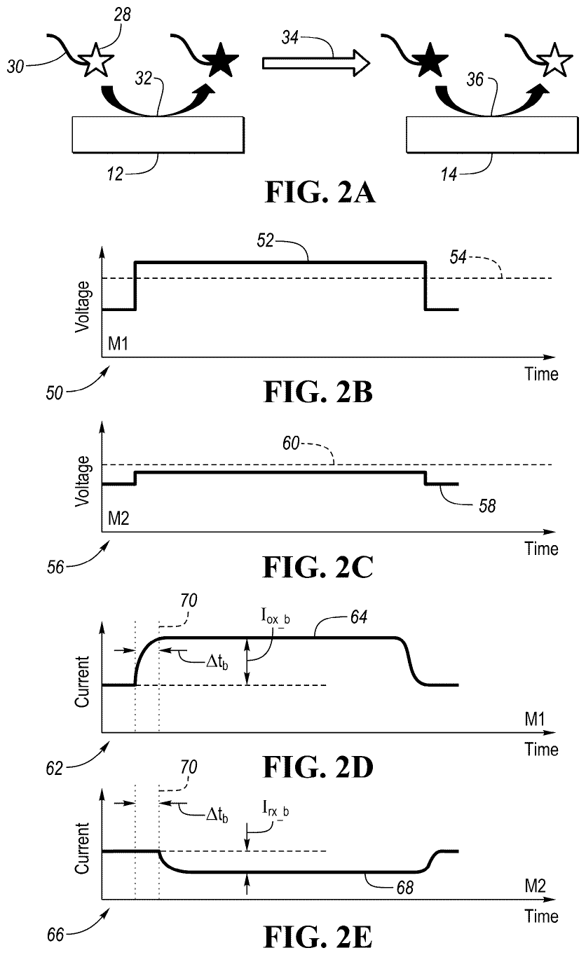

FIG. 2A depicts a schematic of a reaction and diffusion mechanism of redox tags 28 in electrochemical detection system 10. As depicted by first arrow 32, a redox tag 28 in a reduced state changes into an oxidized state at oxidation electrode 12. As depicted by second arrow 34, the redox tag 28 in the oxidized state at oxidation electrode 12 diffuses to reduction electrode 14. At reduction electrode 14, the redox tag 28 in the oxidized state changes to the reduced state as shown by third arrow 36.

FIG. 2B depicts graph 50 of oxidation voltage pulse 52 applied to oxidation electrode 12 (M1) and oxidation potential 54 of oxidation electrode 12. FIG. 2C depicts graph 56 of reduction voltage pulse 58 applied to reduction electrode 14 (M2) and reduction potential 60 of reduction electrode 14. FIG. 2D depicts graph 62 of oxidation current profile 64 at oxidation electrode 12 in response to oxidation voltage pulse 52. FIG. 2E depicts graph 66 of reduction current profile 68 at reduction electrode 14 in response to reduction voltage pulse 58. FIGS. 2B through 2E collectively provide a schematic representation of baseline current measurements according to one embodiment.

As shown in FIGS. 2B and 2C, different potentials (i.e., an oxidation potential and a reduction potential, respectively) are applied to oxidation electrode 12 and reduction electrode 14. A known concentration (e.g., 10 to 100 millimolar range down to a picomolar range) of a capture agent is placed into a solution with a redox tag molecule present. In one case, the redox tag molecule may be in a reduced state. In this case, the capture agent first interacts with the oxidation electrode 12 where the redox tag is oxidized, thereby generating an oxidation current through the oxidation electrode 12. The oxidation current is measured and may be graphed, for example, as shown in FIG. 2D. Some of the capture agent with an oxidized redox tag reaches reduction electrode 14 by diffusion. Some of these oxidized redox tags are reduced at reduction electrode 14, thereby inducing a redox current at reduction electrode 14. The redox current at reduction electrode 14 is measured and may be graphed, for example, as shown in FIG. 2E. The time delay (shown as Δt_{b} 70 in FIGS. 2D and 2E) between an onset of an oxidation current and an onset of a reduction current relates to a diffusion rate of the capture agent. Since there is only one source for the current (i.e., the capture agent), the current profile is in a relatively simple form serving as a baseline signal. One or more electrical parameters may be used to characterize the responses shown in FIGS. 2D and 2E. As described above, one of the electrical parameters may be time delay (Δt_{b}). Other parameters may include oxidation current level in equilibrium (shown as I_{ox_b} in FIG. 2D) and reduction current level in equilibrium (shown as I_{rx_b} in FIG. 2E).

One or more parameters of electrochemical detection system 10 may be optimized to achieve high sensitivity and/or selectivity. The one or more parameters may be an area of oxidation electrode 12 and/or reduction electrode 14, a distance between oxidation electrode and/or reduction electrode 14, a viscosity of the solution, and a concentration of the capture agent.

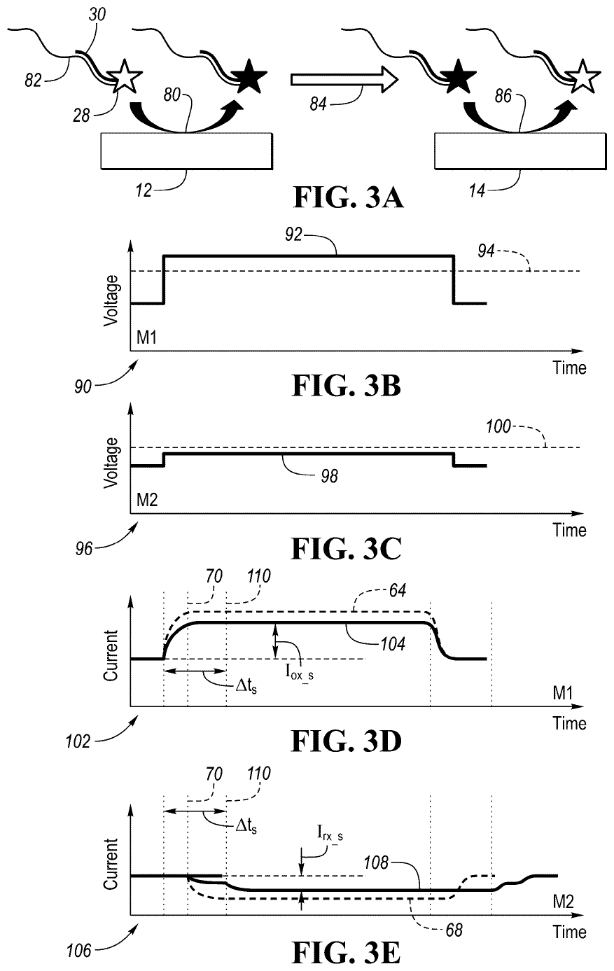

FIG. 3A depicts a schematic view of a reaction and diffusion mechanism of redox tags 28 in electrochemical detection system 10 where target molecules 82 are added to a solution. As depicted by first arrow 80, a redox tag 28 in a reduced state changes into an oxidized state at oxidation electrode 12. As shown in FIG. 3A, capture agent 30 and target molecule 82 are bound to redox tag 28. As depicted by second arrow 84, the redox tag 28 in the oxidated state at oxidation electrode 12 diffuses to reduction electrode 14. At reduction electrode 14, the redox tag 28 in the oxidized state changes to the reduced state as shown by third arrow 86.

FIG. 3B depicts graph 90 of oxidation voltage pulse 92 applied to oxidation electrode 12 (M1) and oxidation potential 94 of oxidation electrode 12. FIG. 3C depicts graph 96 of reduction voltage pulse 98 applied to reduction electrode 14 (M2) and reduction potential 100 of reduction electrode 14. FIG. 3D depicts graph 102 of oxidation current profile 104 at oxidation electrode 12 in response to oxidation voltage pulse 92. FIG. 3D also depicts the baseline oxidation current measurement (i.e., oxidation current profile 64). FIG. 3E depicts graph 106 of reduction current profile 108 at reduction electrode 14 in response to reduction voltage pulse 98. FIG. 3E also depicts the baseline reduction current measurement (i.e., reduction current profile 68).

When target molecules 82 are added to a solution, some of the capture agents 30 bind to the target molecules 82, thereby reducing the diffusion rate between oxidation electrode 12 and reduction electrode 14. In this case, the same procedure used for baseline signal detection as shown in FIGS. 2A through 2E may be used to detect a change introduced by the target molecules. In this case, there is a mixed population of unbound capture agents and bound complexes of the target and the capture agents. The mixed population increases the complexity of the current response as compared to the baseline measurement case. Under this case, unbound capture agents introduce an onset of a current with the same time delay as the baseline measurement case but in a smaller amount due to the decreased concentration. The bound complex contributes to a second onset of a current appearing with a time delay (shown as Δtₛ 110 in FIGS. 3D and 3E). One or more electrical parameters may be used to characterize the responses shown in FIGS. 3D and 3E. As described above, one of the electrical parameters may be time delay (Δtₛ). Other parameters may include oxidation current level in equilibrium (shown as I_{ox_s} in FIG. 3D) and reduction current level in equilibrium (shown as I_{rx_s} in FIG. 3E). In one or more embodiments, the difference between Δtₛ and Δt_{b}, I_{ox_s} and I_{ox_b}, and/or I_{rx_s} and I_{rx_b} may be used to determine the presence of the target molecules. In one or more embodiments, the induced change in the electrochemical signal indicates the presence of the target molecules and quantifying the amount of target molecules may be accomplished through the deconvolution of the signals.

The sensing mechanism of the electrochemical detection systems of one or more embodiments are dependent on a diffusion rate change. In these embodiments, it is beneficial to minimize disturbances in a sample solution containing the target molecules. One embodiment for minimizing disturbances is to confine a measurement volume of the electrochemical detection system.

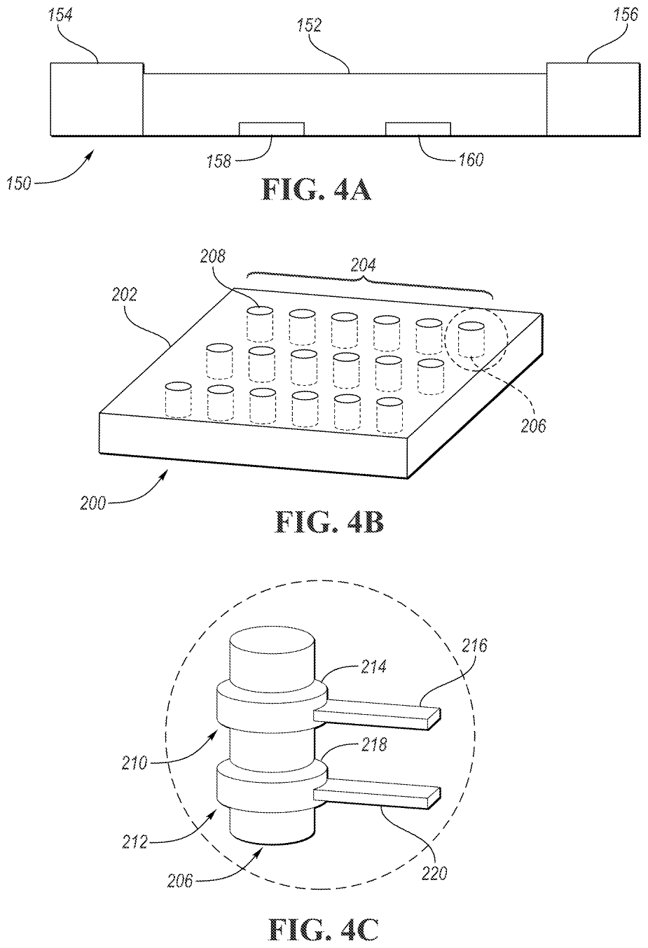

FIG. 4A depicts a schematic view of electrochemical detection system 150 including microchannel 152 partially enclosed first valve 154 and second valve 156 connected thereto. Microchannel 152 is configured to house a relatively small volume of the sample solution (e.g., picoliter range, nanoliter range, or microliter range). The relatively small volume of sample solution increases sensitivity because the target molecules have a higher likelihood to interact with both oxidation electrode 158 and reduction electrode 160 during multiple rounds of measurements. First valve 154 and second valve 156 are configured to confine the sample solution to microchannel 152 to minimize disturbances in the sample solution.

FIG. 4B depicts a schematic, representative view of electrochemical detection system 200 including base 202 and array of microwells 204 extending into base 202. FIG. 4C depicts a magnified view of microwell 206 in the array of microwells 204. As shown in FIG. 4B, the microwell has a cylindrical shape. The microwell structure is configured to increase a retention time of the molecules therein within a relatively small volume. In one or more embodiments, openings 208 of microwells 204 may have a mechanical cover and/or be covered with a non-miscible liquid (e.g., oil). As shown in FIG. 4B, oxidation electrode 210 and reduction electrode 212 are arranged to contact the interior volume of microwell 206. Oxidation electrode 210 and reduction electrode 212 are spaced apart from each other by a fixed distance. Reduction electrode 212 is spaced apart from the bottom of microwell 206 by a fixed distance. Oxidation electrode 210 is spaced apart from the opening of microwell 206 by a fixed distance. Oxidation electrode 210 is closer to the opening of microwell 206 than reduction electrode 212. In an alternative embodiment, reduction electrode 212 is closer to the opening of microwell 206 than oxidation electrode 210. Oxidation electrode 210 includes base portion 214 having a profile (e.g., a circular profile as shown in FIG. 4C) and lead 216 extending from base portion 214. An inner surface portion of base portion 214 contacts the solution within microwell 206. Lead 216 is connected to an electrical source. Reduction electrode 212 includes base portion 218 having a profile (e.g., a circular profile as shown in FIG. 4C) and lead 220 extending from base portion 218. An inner surface portion of base portion 218 contact the solution within microwell 206. Lead 220 is connected to the electrical source.

The processes, methods, or algorithms (e.g., detecting the target molecules in response to one or more electrical properties of the first and second oxidation current profiles and the first and second reduction current profiles) disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A target molecule detection system comprising:
a chamber configured to contain a solution having redox tags and capture agents bound to the redox tags, the redox tags are present in the solution in a reduced state and/or an oxidized state, the capture agents configured to bind to target molecules introduced into the solution;
an oxidation electrode configured to oxidize the redox tags present in the solution in the reduced state into the oxidized state, the oxidation electrode configured to output a first oxidation current profile in response to a first oxidation voltage pulse applied to the oxidation electrode when the solution does not contain the target molecules, the oxidation electrode configured to output a second oxidation current profile in response to a second oxidation voltage pulse applied to the oxidation electrode when the solution contains the target molecules; and
a reduction electrode spaced apart from the oxidation electrode and configured to reduce the redox tags present in the solution in the oxidized state into the reduced state, the reduction electrode configured to output a first reduction current profile in response to a first reduction voltage pulse applied to reduction electrode when the solution does not contain the target molecules, the reduction electrode configured to output a second reduction current profile in response to a second reduction voltage pulse applied to the reduction electrode when the solution contains the target molecules, one or more electrical properties of the first and second oxidation current profiles and the first and second reduction current profiles are indicative of a presence of the target molecules.

2. The target molecule detection system of claim 1, wherein a signaling process is configured to start from the oxidation electrode when the redox tag is stable in the reduced state as an initial state, and the signaling process is configured to start from the reduction electrode when the redox tag is stable in the oxidized state as the initial state.

3. The target molecule detection system of claim 1, wherein the first and second oxidation voltage pulses are equal and the first and second reduction voltage pulses are equal, and the first and second oxidation pulses are different than the first and second reduction pulses.

4. The target molecule detection system of claim 1, wherein the first and second oxidation voltage pulses are greater than an oxidation potential of the redox tag molecule.

5. The target molecule detection system of claim 1, wherein the first and second reduction voltage pulses are less than a reduction potential of the redox tag molecule.

6. The target molecule detection system of claim 1, wherein the oxidation electrode and the reduction electrode are spaced apart a fixed distance.

7. The target molecule detection system of claim 1, wherein the one or more electrical properties of the first and second oxidation current profiles includes a time delay onset oxidation current and a steady state oxidation current level.

8. The target molecule detection system of claim 1, wherein the one or more electrical properties of the first and second reduction current profiles includes a time delay onset reduction current and a steady state reduction current level.

9. The target molecule detection system of claim 1, wherein the solution has a first redox tag concentration when the solution does not contain the target molecules and a second redox tag concentration when the solution contains the target molecules, and the first and second redox tag concentrations are equal.

10. The target molecule detection system of claim 1, wherein the one or more electrical properties of the first and second oxidation current profiles and the first and second reductions current profiles are indicative of an amount of the target molecules.

11. A target molecule detection method comprising:
contacting an oxidation electrode and a reduction electrode with a solution having redox tags and capture agents bound to the redox tags, the redox tags present in the solution in a reduced state and/or an oxidized state, the capture agents configured to bind to target molecules introduced into the solution;
applying a first oxidation voltage pulse to the oxidation electrode when the solution does not contain the target molecules to obtain a first oxidation current profile;
applying a second oxidation voltage pulse to the oxidation electrode when the solution contains the target molecules to obtain a second oxidation current profile;
applying a first reduction voltage pulse to the reduction electrode when the solution does not contain the target molecules to obtain a first reduction current profile;
applying a second reduction voltage pulse to the reduction electrode when the solution contains the target molecules to obtain a second reduction current profile; and
detecting the target molecules in response to one or more electrical properties of the first and second oxidation current profiles and the first and second reduction current profiles.

12. The target molecule detection method of claim 11, wherein a signaling process is configured to start from the oxidation electrode when the redox tag is stable in the reduced state as an initial state, and the signaling process is configured to start from the reduction electrode when the redox tag is stable in the oxidized state as the initial state.

13. The target molecule detection method of claim 11, wherein the first and second oxidation voltage pulses are equal and the first and second reduction voltage pulses are equal, and the first and second oxidation pulses are different than the first and second reduction pulses.

14. The target molecule detection method of claim 11, wherein the first and second oxidation voltage pulses are greater than an oxidation potential of the redox tag molecule.

15. The target molecule detection method of claim 14, wherein the first and second reduction voltage pulses are less than a reduction potential of the redox tag molecule.

16. The target molecule detection method of claim 15, wherein the oxidation potential of the redox tag molecule is greater than the reduction potential of the redox tag molecule.

17. The target molecule detection method of claim 15, further comprising maintaining a redox tag concentration when the solution does not contain the target molecules and when the solution contains the target molecules.

18. The target molecule detection method of claim 15, further comprising deconvoluting the first and second oxidation current profiles and the first and second reduction current profiles to obtain an amount of the target molecules.

19. The target molecule detection method of claim 11, wherein the first, second, third, and fourth applying steps are performed without replacing the solution or washing the oxidation electrode or the reduction electrode.

20. A target molecule detection method comprising:
contacting an oxidation electrode and a reduction electrode with a solution having redox tags and capture agents bound to the redox tags, the redox tags present in the solution in a reduced state and/or an oxidized state, the capture agents configured to bind to target molecules introduced into the solution;
applying a first oxidation voltage pulse to the oxidation electrode when the solution does not contain the target molecules to obtain a first oxidation current profile;
applying a second oxidation voltage pulse to the oxidation electrode when the solution contains the target molecules to obtain a second oxidation current profile;
applying a first reduction voltage pulse to the reduction electrode when the solution does not contain the target molecules to obtain a first reduction current profile;
applying a second reduction voltage pulse to the reduction electrode when the solution contains the target molecules to obtain a second reduction current profile; and
detecting the target molecules in response to a first difference between the first and second oxidation current profiles and/or a second difference between the first and second reduction current profiles.

21. The target molecule detection method of claim 20, wherein a signaling process is configured to start from the oxidation electrode when the redox tag is stable in the reduced state as an initial state, and the signaling process is configured to start from the reduction electrode when the redox tag is stable in the oxidized state as the initial state.

22. The target molecule detection method of claim 20, wherein the detecting step includes detecting the target molecules in response to the first difference between the first and second oxidation current profiles and/or the second difference between the first and second reduction current profiles.

23. The target molecule detection method of claim 20, wherein the oxidation electrode and the reduction electrode include a first electrode pair and a second electrode pair, and further comprising repeating the first, second, third, and fourth applying steps for each of the first electrode pair and the second electrode pair.
